(19) United States
(12) Patent Application Publication
(10) Pub. No.: US 2008/0044358 A1
(43) Pub. Date: Feb. 21, 2008

(54) METHODS FOR LYMPH SYSTEM IMAGING

(76) Inventors: Vincent Jacques, Somerville, MA (US); Peter D. Caravan, Cambridge, MA (US); Bernd Misselwitz, Glienicke (DE); Hanns-Joachim Weinmann, Berlin (DE)

Correspondence Address: FISH & RICHARDSON P.C., PO BOX 1022, MINNEAPOLIS, MN, 55440-1022, US

(21) Appl. No.: 11/840,070

(22) Filed: Aug. 16, 2007

Related U.S. Application Data
(60) Provisional application No. 60/838,488, filed on Aug. 17, 2006.

(51) Int. Cl. A61B 5/055; A61P 35/00

(52) U.S. Cl. 424/9.3

(57) ABSTRACT

Methods and contrast agents for imaging the lymph system are provided. The methods allow the diagnosis and staging of diseases of the lymph system, such as cancer and infections.

### CROSS-REFERENCE TO RELATED APPLICATION

This application claims the benefit under 35 U.S.C. § 119 of the filing date of U.S. Provisional Patent Application Ser. No. 60/838,488, filed on Aug. 17, 2006, the entire contents of which are hereby incorporated by reference.

### TECHNICAL FIELD

This invention relates to imaging of the lymph system, including imaging of lymph nodes and lymph ducts, e.g., for the diagnosis of diseases such as primary and metastatic cancers.

### BACKGROUND

The diagnosis and staging of most cancers, such as breast, lung, head and neck, bladder, kidney, skin, rectal and prostate cancers, often require removal and pathological examination of lymph tissue. For example, approximately 65% of women with breast cancer have a cancer that has spread (metastasized) to the lymph nodes in the nearby area of the original cancer. Physical examination and diagnostic imaging procedures can be unreliable for determining whether or not the cancer has metastasized to the lymph nodes, and surgical removal of regional lymph nodes is often required for accurate cancer staging. For those patients with cancerous lymph nodes, systemic chemotherapy, radiation therapy and/or surgery is currently necessary for control of regional disease.

Lymph nodes are characterized primarily by their size. Enlarged lymph nodes, relative to a standardized size criterion for the nodal region, are often assumed to be a result of tumor invasion. It is well established, however, that there are often small tumor deposits in nodes of normal size, and that over 30% of enlarged nodes contain no tumor and are enlarged solely as a result of inflammation. Other morphological characteristics can be taken into account, such as nodal shape, location, number of nodes, and signal attenuation/enhancement patterns, but diagnostic accuracy is typically low. Thus, there is a need for a more specific identification of cancer as distinguished from inflammation and related physiologies, e.g., benign hyperplasia of lymph nodes.

Ultrasmall preparations of iron oxide (USPIO) have been shown to be suitable as MR contrast agents for intravenous MR lymph node imaging.¹⁻⁴ These USPIO, such as AMI-227 (Combidex®, Sinerem®), have a long plasma circulation time. The particles are gradually taken up by macrophages and transported through the lymphatic system to the lymph nodes. Once the particles have accumulated in the nodes, the high iron content results in a strong T2* susceptibility effect, which serves to make the normal lymph node appear dark on a T2-weighted image. If the lymph node contains tumor cells, then the USPIO are not taken up to the same extent. Thus, the metastases appear bright relative to the normal lymph nodes, and diagnostic accuracy is greatly improved with the use of this contrast agent. One drawback of this approach, however, is that it requires the patient to be imaged prior to injection of the USPIO and then to be imaged again 24-36 hours post injection, which can be inconvenient and may result in poor patient compliance. There may also be difficulties in co-registering the two sets of images, as the positioning of the patient each time will necessarily be different.

Another approach has been to administer the MRI contrast agent interstitially, akin to the nuclear medicine technique of lymphangiography. There have been several reports using animal models where a gadolinium complex is injected interstitially into tissue.⁵⁻²¹ The gadolinium complex then drains into the lymphatic system and moves from lymph node to lymph node until returning to the blood circulation by draining into the subclavian vein through the thoracic duct. This methodology makes the lymph vessels and normal lymph nodes appear bright on a T1-weighted MR image. If there is tumor invasion in a node, the tumor would be seen as a (dark) void in the image, i.e., it would not enhance. Interstitial injection is useful for identifying the sentinel lymph node of a primary tumor. For general imaging of lymph nodes, however, it suffers the drawback of limited distribution, because the gadolinium complex will only enhance the lymph nodes along its path of drainage. In addition, depending on the location of the primary tumor, it may also be difficult to administer the agent interstitially to an area that enhances the lymph nodes of interest.

Two other MR approaches have been reported. One involved conjugating a gadolinium complex to a glucose containing polymer;²² localization in the lymph nodes, however, was slow (˜24 hours). Another approach utilized a gadolinium complex containing a perfluorocarbon chain.²³ Lymph nodes enhanced in 15 minutes post i.v. injection in a rabbit model and tumor bearing lymph nodes could be distinguished from normal nodes.

There remains a need for a method that can quickly and systemically image lymph nodes; that can identify the presence of cancer and metastatic cancer; and that can provide a differential diagnosis of cancer/metastatic disease from inflammation. Such an agent could have a significant clinical impact in reducing the need for diagnostic surgical lymph node removal and its attendant complications.

### SUMMARY

The disclosure is directed to the finding that certain MR contrast agents, such as those that include a phosphodiester moiety and that can bind to a plasma protein, such as human serum albumin (HSA), are useful for imaging of the lymph system. Use of the contrast agents can allow better diagnosis, staging, and subsequent treatment of many cancers, as well as the diagnosis and treatment of other lymph system diseases, including parasitic infections and Castleman disease. Finally, use of the contrast agents can allow the differential diagnosis of cancer/metastatic disease from inflammation, infection, or benign hyperplasia of the lymph nodes.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A, B, and C demonstrate imaging of metastatic iliacal lymph nodes (arrows) in a rabbit, acquired with a T1-weighted gradient-echo sequence at 5 min. after intravenous injection of 0.2 mmol/kg of Gd-DTPA (A), and 15 min. after intravenous injection of 0.05 mmol/kg of MS-325 (B), respectively, in the same animal. A histological section (C) with hematoxylin-eosin staining of the same lymph nodes (M: metastases) is also shown.

FIGS. 2 A, B, and C demonstrates imaging of metastatic iliacal lymph nodes (arrows) of a rabbit, acquired with a T1-weighted gradient-echo sequence at 5 min. after intravenous injection of 0.2 mmol/kg of Gd-DTPA (A), and 0.05 mmol/kg of MS-325 (B), respectively, in the same animal. A histological section (C) with hematoxylin-eosin staining of the same lymph nodes (M: metastases) is also shown.

### DETAILED DESCRIPTION

### Definitions

In general, the term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, such as naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles,” “heterocycles,” “heteroaryls,” or “heteroaromatics.” An aryl group may be substituted at one or more ring positions with substituents.

For the purposes of this application, “DTPA” refers to a chemical compound comprising a substructure composed of diethylenetriamine, wherein the two primary amines are each covalently attached to two acetyl groups and the secondary amine has one acetyl group covalently attached according to the following formula:

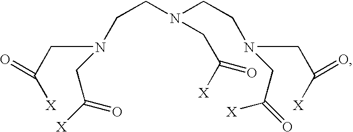

wherein X is a heteroatom electron-donating group capable of coordinating a metal cation, preferably O⁻, OH, NH₂, OPO₃²⁻, or NHR, or OR wherein R is any aliphatic group. When each X group is tert-butoxy (tBu), the structure may be referred to as “DTPE” (“E” for ester).

For the purposes of this application, “DOTA” refers to a chemical compound comprising a substructure composed of 1,4,7,11-tetraazacyclododecane, wherein the amines each have one acetyl group covalently attached according to the following formula;

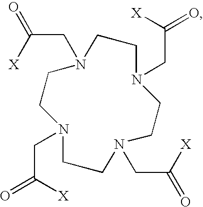

wherein X is defined above.

For the purposes of this application, “NOTA” refers to a chemical compound comprising a substructure composed of 1,4,7-triazacyclononane, wherein the amines each have one acetyl group covalently attached according to the following formula:

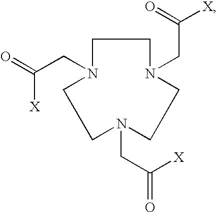

wherein X is defined above.

For the purposes of this application, “DO3A” refers to a chemical compound comprising a substructure composed of 1,4,7,11-tetraazacyclododecane, wherein three of the four amines each have one acetyl group covalently attached and the other amine has a substituent having neutral charge according to the following formula:

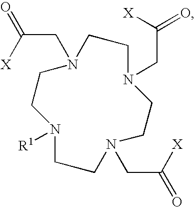

wherein X is defined above and R¹ is an uncharged chemical moiety, preferably hydrogen, any aliphatic, alkyl group, or cycloalkyl group, and uncharged derivatives thereof. The preferred chelate “HP”-DO3A has R¹=—CH₂(CHOH)CH₃.

In each of the four structures above, the carbon atoms of the indicated ethylenes may be referred to as “backbone” carbons. The designation “bbDTPA” may be used to refer to the location of a chemical bond to a DTPA molecule (“bb” for “back bone”). Note that as used herein, bb(CO)DTPA-Gd means a C═O moiety bound to an ethylene backbone carbon atom of DTPA.

The terms “chelating ligand,” “chelating moiety,” and “chelate moiety” may be used to refer to any polydentate ligand which is capable of coordinating a metal ion, including DTPA (and DTPE), DOTA, DO3A, or NOTA molecule, or any other suitable polydentate chelating ligand, that is either coordinating a metal ion or is capable of doing so, either directly or after removal of protecting groups, or is a reagent, with or without suitable protecting groups, that is used in the synthesis of a contrast agent and comprises substantially all of the atoms that ultimately will coordinate the metal ion of the final metal complex. The term “chelate” refers to the actual metal-ligand complex, and it is understood that the polydentate ligand will eventually be coordinated to a medically useful metal ion.

The term “specific binding affinity” as used herein, refers to the capacity of a contrast agent to be taken up by, retained by, or bound to a particular biological component to a greater degree than other components. Contrast agents that have this property are said to be “targeted” to the “target” component. Contrast agents that lack this property are said to be “non-specific” or “non-targeted” agents. The specific binding affinity of a binding group for a target is expressed in terms of the equilibrium dissociation constant “Kd.”

The term “relaxivity” as used herein, refers to the increase in either of the MRI quantities 1/T1 or 1/T2 per millimolar (mM) concentration of paramagnetic ion or contrast agent, which quantities may be different if the contrast agent contains a multiplicity of paramagnetic ions, wherein T1 is the longitudinal or spin-lattice, relaxation time, and T2 is the transverse or spin-spin relaxation time of water protons or other imaging or spectroscopic nuclei, including protons found in molecules other than water. Relaxivity is expressed in units of mM⁻¹s⁻¹.

Methods for Imaging the Lymphatic System

In general, methods for MR imaging of the lymphatic system are provided. The methods are useful for a number of reasons, e.g., staging cancer, diagnosing cancer, diagnosing or staging a disease of the lymph system (e.g., infections), guiding biopsies, surgical planning, and therapy monitoring. In addition, the methods can allow one to distinguish between cancer (e.g., a tumor in a lymph node) and normal lymph tissue, fat, and/or inflamed lymph tissue.

In some embodiments of the methods, one or more images of all or a region of the lymphatic system (e.g., a node or collection of nodes) of a mammal is obtained prior to administration of a contrast agent as described herein. A contrast agent is intravascularly injected into a mammal, e.g., into an artery or vein of the mammal, and all or a region of the lymphatic system of the mammal is imaged. The region of the lymphatic system can include one or more lymph nodes, vessels, ducts, channels or combinations thereof, and can be found anywhere in the body of the mammal, e.g., in the iliac, lumbar, inguinal, cervical, axillary, popliteal, cervical and/or neck, mesenteric, or thoracic region of said mammal.

The mammal can be a human, cat, dog, horse, cow, sheep, mouse, rat, rabbit, pig, or monkey. Typically the mammal is a human, e.g., a human patient. In certain cases, the region of the lymphatic system to be imaged has been pre-selected, such as when a mammal is suspected or diagnosed with a cancer of a certain body region. For example, for a human suspected of or diagnosed as having breast cancer, the axillary or supraclavicular lymph system can be pre-selected; or for a human suspected of or diagnosed as having prostate cancer, the pelvic or inguinal lymph system can be preselected. One having ordinary skill in the art would understand the appropriate region to pre-select given a particular diagnosis or suspected disease.

The lymph system or region thereof can be imaged at any time after injection of the contrast agent, e.g., from 1 min. to 24 hours after injection, or any time in between, e.g., 5 min., 10 min., 15 min., 30 min., 45 min., 1 hour, 2 hours, 3 hours, 4 hours, 8 hours, 12 hours, 16 hours, or 20 hours after injection. In some cases, the lymph system or region thereof is imaged at a time from about 5 min. to about 2 hours after injection.

The methods employ the use of an MR contrast agent, which typically enhances normal lymph tissue, but does not enhance cancerous tumors or fat tissue. Certain MR contrast agents for use in the methods include a phosphodiester moiety, a plasma protein binding moiety, and a paramagnetic metal chelate, or a pharmaceutically acceptable salt thereof, where the contrast agent is capable of binding to a plasma protein, as described further herein. Other contrast agents for use in the method include a blood pool contrast agent selected from:

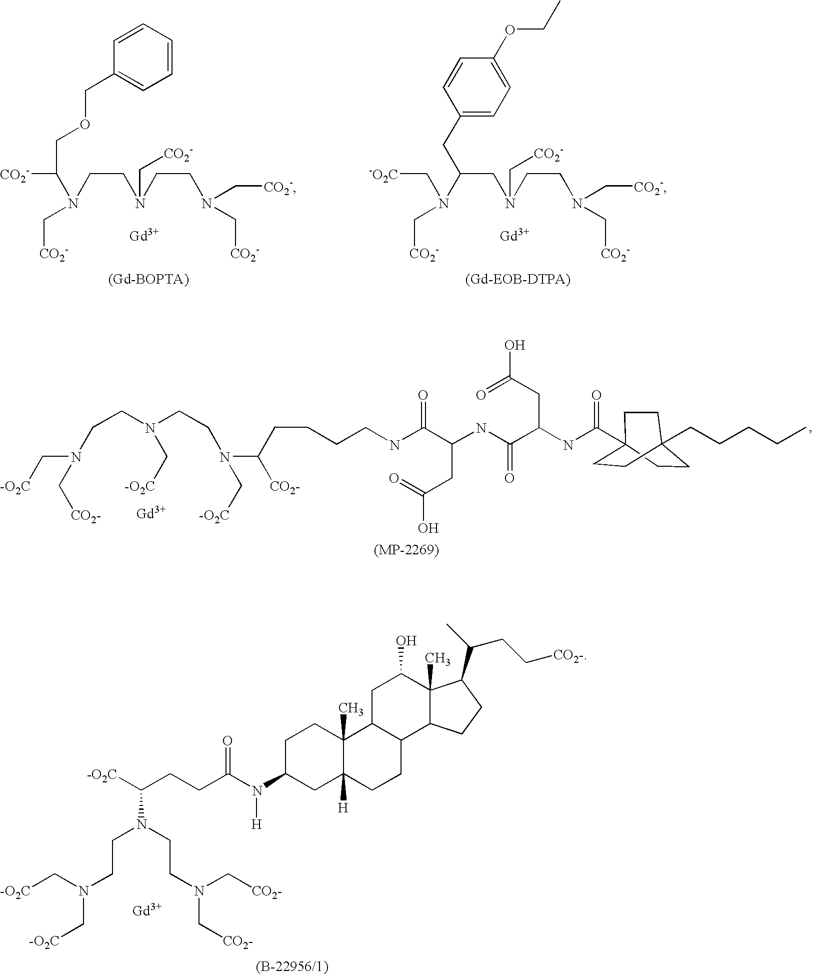

The contrast agents employed in the methods are typically effective with T1-weighted imaging sequences. There are many T1-weighted sequences that are well known to those in the art. These include but are not limited to spin echo sequences with short TR, inversion recovery prepared sequences, and spoiled gradient recalled echo sequences.

Diagnosis and/or staging of a disease such as cancer or metastatic cancerous disease can be based on an evaluation of the MR image signal intensity in the region of the lymphatic system. The evaluation can including a comparison of the signal intensity in the region with the signal intensity from the same (e.g., intra-region comparison, such as comparing signal intensities within a given node) or a different (e.g., inter-region comparison, such as comparing signal intensities of multiple nodes) region. The evaluation can occur before and/or after injection of the contrast agent and associated imaging. The evaluation can include an analysis of how the signal intensity in a given region (e.g., a node) changes (e.g., in absolute amount or a percentage change) after administration of the contrast agent as compared to an image acquired prior to contrast agent administration. For example, in some embodiments, tumors or metastatic cancers present in a lymph node will exhibit hypointensity after contrast agent administration as compared to normal tissue (e.g., normal tissue in the same node or in a different node). This hypointensity is postulated to result because the contrast agent is not taken up into the tumor. The node may appear isointense on the image acquired prior to contrast agent administration, but tumors present within a lymph node can be “dark spots” present in otherwise “bright” nodes after administration of the contrast agent.

In addition, some lymph nodes contain a region of fat within the node referred to as a “fatty hilum”. The fat does not typically enhance with the contrast agents described in this invention. Fat can be distinguished from tumor and/or normal tissue by acquiring an additional image using fat suppression techniques. There are several fat suppression techniques that rely on the difference in resonance frequency between water and fat protons that are well known to those in the art.²⁴ For example, the fat signal can be saturated. On a T1-weighted image, fat would appear bright without fat saturation and would appear dark if the same image was acquired with fat saturation. The signal intensity of normal nodal tissue and tumor would be unchanged between these two scans.

Accordingly, a method for determining the presence or absence of a primary or metastatic cancer in a region of the lymph system can include:

(a) optionally preselecting a region of the lymphatic system of a mammal to image (e.g., a node or collection of nodes);

(b) optionally obtaining a T1-weighted MR image of said region;

(c) intravascularly injecting the mammal with an MR contrast agent, e.g., a blood pool contrast agent as discussed above or a contrast agent comprising a phosphodiester moiety, a PPBM, and a paramagnetic metal chelate, or a pharmaceutically acceptable salt thereof, wherein the contrast agent is capable of binding to a plasma protein; and

(d) obtaining a T1-weighted MR image of said region of the lymphatic system, wherein said determination of the presence or absence of said primary or metastatic cancer is based on an evaluation of the signal intensity in said region of the lymphatic system, as discussed above. In some embodiments, the signal intensity can be evaluated by comparing the image of (d) with the pre-contrast agent image of (b), wherein normal lymph node would show positive signal enhancement post-contrast agent, but the tumor would not significantly enhance.

In some embodiments, the method can further include: (e) optionally obtaining a fat-suppressed T1-weighted MR image of the same region in (d). Differences in signal intensity between the image in (e) and the image in (d) would be due to the presence of fat rather than tumor or normal tissue.

In some embodiments, two or more 2D image planes of the region of the lymphatic system may be examined in order to determine the presence or absence of the primary or metastatic cancer.

Similar methods can be used for guiding a biopsy of a lymph node of a mammal. The method can include:

(a) optionally preselecting a region of the lymphatic system of a mammal to image (e.g., a node or collection of nodes);

(b) optionally obtaining a T1-weighted MR image of said region;

(c) intravascularly injecting the mammal with an MR contrast agent, e.g., a blood pool contrast agent as discussed above or a contrast agent comprising a phosphodiester moiety, a PPBM, and a paramagnetic metal chelate, or a pharmaceutically acceptable salt thereof, wherein the contrast agent is capable of binding to a plasma protein;

(d) obtaining a T1-weighted MR image of said region of the lymphatic system, wherein said determination of the presence or absence of said primary or metastatic cancer is based on an evaluation of the signal intensity in said region of the lymphatic system, as discussed above; and

(e) determining an appropriate location (e.g., a hypointense region) to biopsy, e.g., based on an evaluation of the signal intensity in said region in the MR image of (d), either alone or in comparison to the MR image of (b). The method can further include: (f) optionally obtaining a fat-suppressed T1-weighted MR image of the same region in (d). Differences in signal intensity between the image in (f) and the image in (d) would be due to the presence of fat rather than tumor or normal tissue.

Based on the information provided by the current methods, one of skill in the art could similarly perform methods for guiding and determining the extent of surgery of the lymph system required after diagnosis of cancer; methods for guiding cancer-related lymphadenectomy; methods for monitoring the effectiveness of chemotherapy or radiation on cancer in the lymphatic system; methods for monitoring cancer remittance; and methods for staging cancer.

The present disclosure also provides methods for distinguishing primary cancer and/or metastatic cancer from inflammation of the lymph nodes and/or lymphatic vessels (i.e., lymphadenitis and lymphangitis) and from other non-cancerous diseases of the lymph system, e.g., benign hyperplasia of the lymph nodes, also known as Castleman's disease. Lymph nodes are frequently characterized by their size on a CT or MR image. Depending on the anatomical region, nodes are considered enlarged if they exceed a predetermined size criterion. For example in the mediastinum, lymph nodes with a short axis greater than 1.0 cm are considered enlarged.²⁵ This enlargement may be due to tumor invasion or, for example, to the presence of immune cell activity in the case of an infection. The present disclosure thus provides a method to distinguish a lymph node containing a cancerous tumor from a normal lymph node or from a benign enlarged lymph node (e.g., due to inflammation or benign hyperplasia). The method can include:

(a) optionally preselecting at least one node of the lymphatic system of a mammal to image (e.g., a node or collection of nodes); in some embodiments, the at least one node may have optionally been previously determined to exceed a predetermined size criteria for that anatomical region by a prior CT or MRI scan;

(b) optionally obtaining a T1-weighted MR image of said at least one node;

(c) intravascularly injecting the mammal with an MR contrast agent, e.g., a blood pool contrast agent as discussed above or a contrast agent comprising a phosphodiester moiety, a PPBM, and a paramagnetic metal chelate, or a pharmaceutically acceptable salt thereof, wherein the contrast agent is capable of binding to a plasma protein;

(d) obtaining a T1-weighted MR image of said at least one node, wherein said distinguishing of a node containing a cancerous tumor from a benign enlarged lymph node or from a normal node is based on an evaluation of the signal intensity and/or size of said at least one node. For example, if the at least one node demonstrates relatively uniform enhancement in step (d), then the at least one node can be characterized as either normal or benign reactive. In such a case, the method can include optionally determining the size (e.g., from the MR image of (b) and/or (d)) of said at least one lymph node relative to a predetermined size criterion for that anatomical region. If the size exceeds the predetermined size criterion, then the at least one lymph node can be characterized as benign reactive (e.g., enlarged due to benign hyperplasia or inflammation). In other cases, if the at least one node demonstrates non-uniform enhancement in step (d), such as if there was an hypointense region within the node, then the method can include distinguishing if such an hypointense region was due to the presence of tumor or to the presence of fat. In such embodiments, a fat suppressed T1-weighted image (e) can be acquired:

(e) obtaining a fat suppressed T1-weighted MR image of the at least one node in (d). Differences in signal intensity between this (e) image and the image in (d) would be due to the presence of fat and not tumor.

The current methods are also useful for determining the presence or absence of elephantiasis (parasitic worm infection) in a region of the lymphatic system of a mammal. The method can include:

(a) optionally preselecting a region of the lymphatic system of a mammal to image (e.g., a node or collection of nodes);

(b) optionally obtaining a T1-weighted MR image of said region;

(c) intravascularly injecting the mammal with an MR contrast agent, e.g., a blood pool contrast agent as discussed above or a contrast agent comprising a phosphodiester moiety and a paramagnetic metal chelate, or a pharmaceutically acceptable salt thereof, wherein the contrast agent is capable of binding to a plasma protein;

(d) obtaining a T1-weighted MR image of said region of the lymphatic system, wherein said determination of the presence or absence of said elephantiasis is based on an evaluation of the signal intensity in said region of the lymphatic system.

Contrast Agents for Use in the Methods

Certain of the MR contrast agents for use in the methods can include a phosphodiester moiety and a paramagnetic metal chelate [Chel] and are capable of binding to a plasma protein. Such a contrast agent also includes a plasma protein binding moiety (PPBM) that facilitates the binding to a plasma protein. The phosphodiester moiety can be covalently bound to the chelate, either directly or through a linker, and/or covalently bound to the plasma protein binding moiety, again either directly or through a linker.

Since HSA is present at high concentration in serum (approximately 0.6 mM) and binds a wide array of molecules with reasonably high affinity, it is a preferred target plasma protein for contrast agents; see U.S. Pat. No. 6,676,929, and WO 96/23526. Other useful plasma proteins include fibrinogen, fibrin, alpha acid glycoprotein, globulins, and lipoproteins.

For binding to plasma proteins, a wide range of hydrophobic or amphiphilic substances may be used as the PPBM, including alkyl, cycloalkyl, heteroalkyl, heterocyclyl, aryl, alkaryl, and aralkyl groups having from 1 to 25 carbon atoms, which groups can be optionally substituted with 1 to 5 alkyl, aryl, heteroalkyl, cycloalkyl, heterocycly, alkoxy, hydroxyl, and halo groups. As used herein, the terms “alkyl,” “heteroalkyl,” “cycloalkyl,” and “heterocyclyl” are meant to include unsaturated derivatives that can include from 1 to 3 double and/or triple bonds. Moreover, as used herein, alkyl and heteroalkyl groups can be linear or branched groups.

In certain embodiments, the PPBM can be selected from linear or branched alkyl groups optionally substituted with one or more alkyl (e.g., methyl, ethyl, t-butyl), aryl (e.g., phenyl), alkoxy (e.g., methoxy, ethoxy, t-butoxy) or hydroxyl groups; cycloalkyl groups (e.g., cyclopentyl, cyclohexyl) optionally substituted with one or more alkyl (e.g., methyl, ethyl, t-butyl), aryl (e.g., phenyl), alkoxy (e.g., methoxy, ethoxy, t-butoxy) or hydroxyl groups; or aryl groups (e.g., phenyl) optionally substituted with one or more alkyl (e.g., methyl, ethyl, t-butyl), aryl (e.g., phenyl), alkoxy (e.g., methoxy, ethoxy, t-butoxy) or hydroxyl groups. The PPBM can be covalently conjugated through a phospho-ester linkage to the phosphodiester moiety of the contrast agent.

The paramagnetic metal chelate can be any chelate useful in MR imaging, including, but not limited to DTPA, DOTA, DO3A, and NOTA.

Metal ions preferred for MRI include those with atomic numbers 21-29, 39-47, or 57-83, and, more preferably, a paramagnetic form of a metal ion with atomic numbers 21-29, 42, 44, or 57-83. Particularly preferred paramagnetic metal ions are selected from the group consisting of Gd(III), Fe(III), Mn(II and III), Cr(III), Cu(II), Dy(III), Tb(III and IV), Ho(III), Er(III), Pr(III) and Eu(II and III). Gd(III) is particularly useful. Note that as used herein, the term “Gd” is meant to convey the ionic form of the metal gadolinium; such an ionic form can be written as GD(III), GD3+, gado, etc., with no difference in ionic form contemplated.

In some embodiments, the contrast agent can have a structure as follows:
[Chel]-[Lₘ-{BHEM-PPBM}ₚ]_{q},

or a pharmaceutically acceptable salt or derivative thereof, wherein m, p, and q are, independently, from 1 to 5;
wherein said [Chel] is a paramagnetic metal chelate selected from the group consisting of: 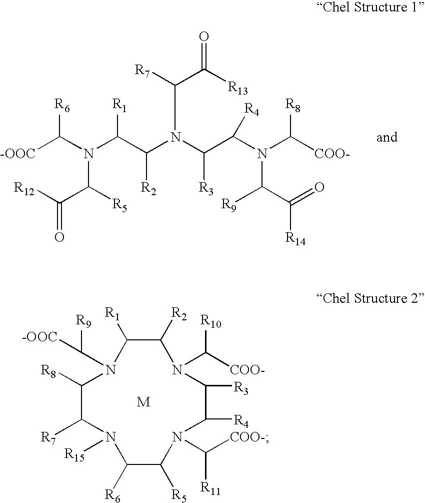
wherein at least one of said R₁-R₁₁ is -[Lₘ-{BHEM-PPBM}ₚ] and the R₁-R₁₁ groups that are not -[Lₘ-{BHEM-PPBM}ₚ] are selected from hydrogen and C1-C4 alkyl;
wherein R₁₂, R₁₃, and R₁₄ can be the same or different and are selected from the group consisting of O⁻, and NH₂;
wherein R₁₅ is H, CH₂CH(OH)CH₃, hydroxyalkyl, or CH₂COR₁₂;
wherein said M is a paramagnetic metal ion selected from the group consisting of Gd(III), Fe(III), Mn(II), Mn(III), Cr(III), Cu(II), Dy(III), Tb(III), Ho(III), Er(III), and Eu(III);
wherein said L is a linker as described below;
wherein said BHEM is said phosphodiester moiety; and
wherein said PPBM is a plasma protein binding moiety, as described previously.

In some embodiments, m, p, and q are each 1.

In some embodiments, only 1 of said R₁-R₁₁ groups is -[Lₘ-{BHEM-PPBM}ₚ] and the R₁-R₁₁ groups that are not -[Lₘ-{BHEM-PPBM}ₚ] are hydrogen.

In some embodiments, R₁₂, R₁₃, and R₁₄ are O⁻.

In some embodiments, R₁₅ is H.

In some embodiments, M is Gd(III).

In some embodiments, L is —(CH₂)ₙ—, wherein n is from 1 to 5.

In some embodiments, the PPBM is selected from alkyl, cycloalkyl, heteroalkyl, heterocyclyl, aryl, alkaryl, and aralkyl groups having from 1 to 25 carbon atoms, wherein said groups can be optionally substituted with 1 to 5 alkyl, aryl, heteroalkyl, cycloalkyl, heterocycly, alkoxy, hydroxyl, and halo groups.

In some embodiments, the PPBM is selected from linear or branched alkyl groups optionally substituted with one or more alkyl, aryl, alkoxy or hydroxyl groups; cycloalkyl groups optionally substituted with one or more alkyl, aryl, alkoxy or hydroxyl groups; and aryl groups optionally substituted with one or more alkyl, aryl, alkoxy or hydroxyl groups.

Some preferred phosphodiester-containing contrast agents include those having the following structures: 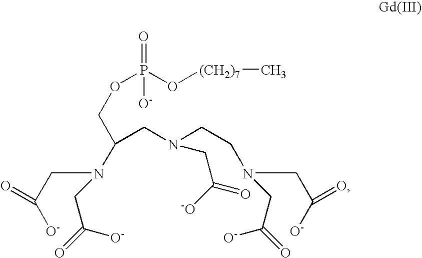
referred to as MS-315; 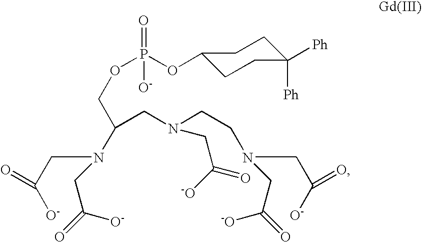
wherein Ph=phenyl, referred to as MS-325; 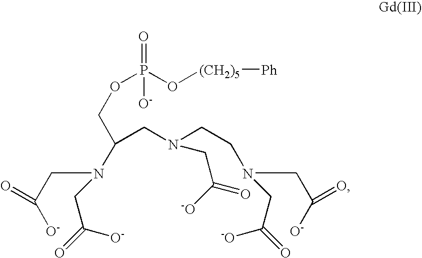
wherein Ph=phenyl; referred to as MS 317; 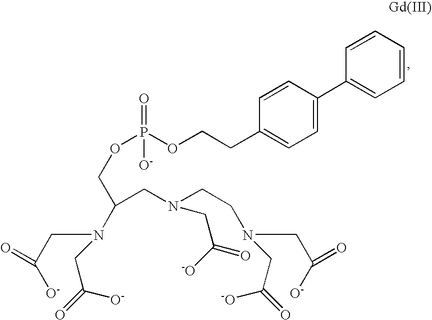
referred to as MS-322; 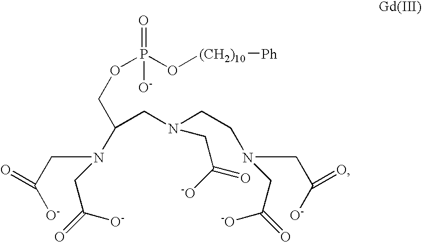
wherein Ph=phenyl, referred to as MS-323; 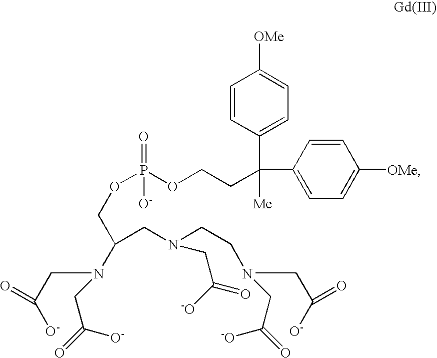
wherein Me=methyl, referred to as MS-328; 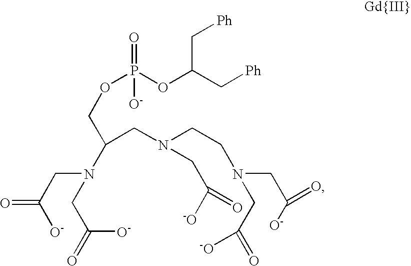
wherein Ph=phenyl, referred to as MS-326; and 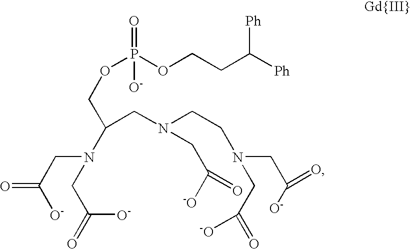
wherein Ph=phenyl; referred to as MS-327.

The above contrast agents are further described in U.S. Pat. No. 6,676,929, incorporated by reference herein.

Other contrast agents for use in the method include a blood pool contrast agent selected from: 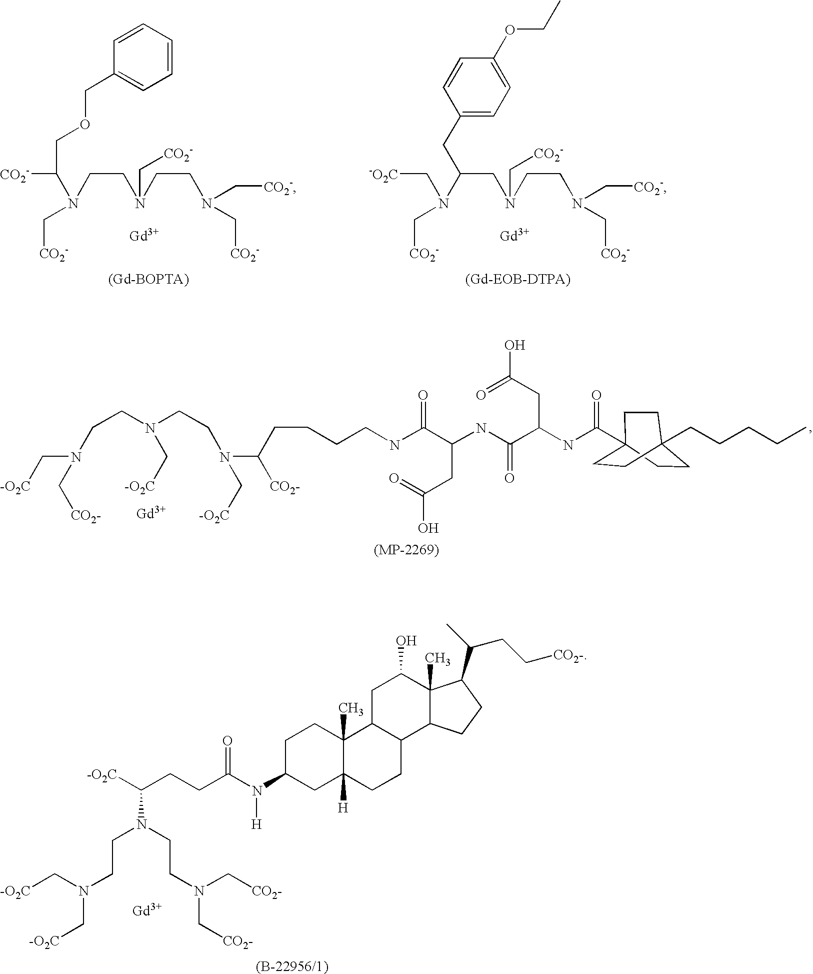
Linker Moieties

The phosphodiester moiety, chelate, and plasma protein binding moiety can be directly bonded to each other. Alternatively, they can be joined through a linker L. The linker can be peptidic or non-peptidic in nature. The linker can be an alkyl group (e.g., a methylene chain having from 1 to 10 carbon atoms (1, 2, 3, 4, 5, 6, 7, 8, 9 or 10 carbon atoms), or can contain heteroatoms such as, e.g., oxygen, nitrogen, sulfur, and phosphorus. The linker can contain a PEG (polyether) region. The linker can be a linear or branched chain, or can include structural elements such as phenyl ring(s), non-aromatic carbocyclic or heterocyclic ring(s), double or triple bond(s), and the like. Linkers may be substituted with alkyl or aryl groups. The linker moieties can include multiple functional groups, which can be conjugated to one or more chelates, phosphodiester moieties, or PPBM moieties. Preferred linkers include alkyl groups having from 1 to 5 —CH₂— groups, e.g., —(CH₂)ₙ—, wherein n can be 1 to 5.

Properties of Contrast Agents

Contrast agents of the invention can bind a plasma protein target such as human serum albumin. For example, at least 10% (e.g., at least 50%, 80%, 90%, 92%, 94%, or 96%) of the contrast agent can be bound to the desired target at physiologically relevant concentrations of drug and target. The extent of binding of a contrast agent to a target, such as HSA, can be assessed by a variety of equilibrium binding methods. For example, binding to HSA can be measured by ultrafiltration. The concentration of bound contrast agent is determined as the difference between the total targeting group concentration initially present and the unbound targeting group concentration following the binding assay. The bound fraction is the concentration of bound targeting group divided by the concentration of total targeting group.

Compounds of the invention can exhibit high relaxivity as a result of target binding (e.g., to HSA), which can lead to better image resolution. The increase in relaxivity upon binding is typically 1.5-fold or more (e.g., at least a 2, 3, 4, 5, 6, 7, 8, 9, or fold increase in relaxivity). Targeted contrast agents having 7-8 fold, 9-10 fold, or even greater than 10 fold increases in relaxivity are particularly useful. Typically, relaxivity is measured using an NMR spectrometer. The preferred relaxivity of an MRI contrast agent at 20 MHz and 37° C. is at least 10 mM-1s-1 per paramagnetic metal ion (e.g., at least 15, 20, 25, 30, 35, 40, or 60 mM-is-1 per paramagnetic metal ion. Contrast agents having a relaxivity greater than 60 mM-1s-1 at 20 MHz and 37° C. are particularly useful.

MR Techniques

Contrast agents prepared according to the disclosure herein may be used in the same manner as conventional MRI contrast agents and are useful for the diagnosis and staging of cancer and lymph system infections, inflammation, and disorders (e.g., Castleman disease). As described herein, the presently described plasma-protein targeted contrast agents can show an increase in lymph node uptake relative to other contrast agents. In addition, tumor containing (cancerous) lymph tissue can appear hypointense relative to normal (e.g., normal) or benign enlarged (e.g. infected) lymph tissue. Specificity of uptake of plasma-protein-targeted contrast agents by the lymph system can be can be demonstrated using MRI and observing relative enhancement (e.g., signal intensity) of lymph system signal.

When imaging a region of the lymph system (e.g., a node), certain MR techniques and pulse sequences may be preferred to enhance the contrast of normal lymph tissue as compared to cancerous tissue. These techniques include, but are not limited to, T1-weighted images, such as inversion-recovery prepared, or saturation-recovery prepared, or spoiled gradient recalled echo, or spin echo sequences that will increase the contrast between the enhanced normal (or benign reactive) lymph tissue and tumor. Methods of preparation for T2 techniques may also prove useful. Finally, preparations for magnetization transfer techniques may also improve contrast with agents of the invention.

Pharmaceutical Compositions

Contrast agents can be formulated as pharmaceutical compositions in accordance with routine procedures. As used herein, the compounds of the invention can include pharmaceutically acceptable salts or derivatives thereof. “Pharmaceutically acceptable” means that the compound or composition can be administered to an animal without unacceptable adverse effects. A “pharmaceutically acceptable derivative” means any pharmaceutically acceptable salt, ester, salt of an ester, or other derivative of a compound of this invention that, upon administration to a recipient, is capable of providing (directly or indirectly) a compound of this invention or an active metabolite or residue thereof. Pharmaceutically acceptable salts of the compounds of this invention include counter ions derived from pharmaceutically acceptable inorganic and organic acids and bases known in the art.

Pharmaceutical compositions of the invention can be administered parenterally by intravenous or intra-arterial administration. When administration is intravenous, pharmaceutical compositions may be given as a bolus, as two or more doses separated in time, or as a constant or non-linear flow infusion.

Typically, compositions for administration are solutions in sterile isotonic aqueous buffer. Where necessary, the composition may also include a solubilizing agent, a stabilizing agent, and a local anesthetic such as lidocaine to ease pain at the site of the injection. Generally, the ingredients will be supplied either separately, e.g. in a kit, or mixed together in a unit dosage form, for example, as a dry lyophilized powder or water free concentrate. The composition may be stored in a hermetically sealed container such as an ampule or sachette indicating the quantity of active agent in activity units. Where the composition is administered by infusion, it can be dispensed with an infusion bottle containing sterile pharmaceutical grade “water for injection,” saline, or other suitable intravenous fluids. Where the composition is to be administered by injection, an ampule of sterile water for injection or saline may be provided so that the ingredients may be mixed prior to administration. Pharmaceutical compositions of this invention comprise the compounds of the present invention and pharmaceutically acceptable salts thereof, with any pharmaceutically acceptable ingredient, excipient, carrier, adjuvant or vehicle.

A contrast agent is preferably administered to the patient in the form of an injectable composition. The method of administering a contrast agent is preferably intravenously or intra-arterially. As described previously, intravenous administered can be preferred. Pharmaceutical compositions of this invention can be administered to mammals including humans in a manner similar to other diagnostic or therapeutic agents. The dosage to be administered, and the mode of administration will depend on a variety of factors including age, weight, sex, condition of the patient and genetic factors, and will ultimately be decided by medical personnel subsequent to experimental determinations of varying dosage followed by imaging as described herein. In general, dosage required for diagnostic sensitivity or therapeutic efficacy will range from about 0.001 to 50,000 μg/kg, preferably between 0.01 to 25.0 μg/kg of host body mass. The optimal dose will be determined empirically following the disclosure herein.

### EXAMPLES

### Example 1

### Detection of Lymph Node Metastases in a VX2 Tumor Rabbit Model After Single Intravenous Injection of MS-325: Comparison with Gd-DTPA

Objectives:

The purpose of this study was to demonstrate the lymph node enhancement and the detection of lymph node metastases after intravenous injection of the contrast agent MS-325 in comparison with an extracellular, non-plasma protein targeted contrast agent Gd-DTPA.

Materials and Methods:

Animal Models:

All experimental protocols were performed in accordance with applicable regulations governing animal experiments.

VX2 Tumor Bearing Rabbits:

New Zealand White rabbits (3-4 kg, n=6) were inoculated intramuscularly into the thigh with 2 to 3 pieces (1×1 mm) of VX2 carcinoma cells to produce metastases in iliacal lymph nodes. The imaging experiments were performed 3 to 6 weeks after the injection of tumor cells.

MR Imaging:

MR system: Head scanner (Allegra, 1.5 Tesla; Siemens AG, Erlangen, Germany), T1-weighted sequence (3D-vibe, TR/TE 3.74/1.71 ms, a 20°, slice thickness 1 mm).

Contrast agents: Gd-DTPA (0.2 mmol Gd/kg), MS-325 (0.05 mmol Gd/kg).

Imaging: Intraindividual comparison of lymphographic effects in iliacal lymph nodes.

day 1: Gd-DTPA (5 to 120 minutes p.i.).

day 2: MS-325 (5 to 120 minutes p.i.).

Analysis:

Assessment of technical success and quality of MR imaging, and detection of lymph node metastases.

Histology: Microscopic examination after H/E staining; correlation with MR findings.

Results:

MR imaging of VX2 tumor bearing rabbits revealed a rapid and strong signal increase in the functional lymph node tissue between 5 and 30 min after intravenous injection of MS-325. The metastatic tissue showed only a slight enhancement resulting in an excellent delineation of the lymph node metastases. In contrast, Gd-DTPA induced only a slight and inhomogeneous enhancement in the whole lymph node, which does not allow an effective differentiation of functional and metastatic tissue.

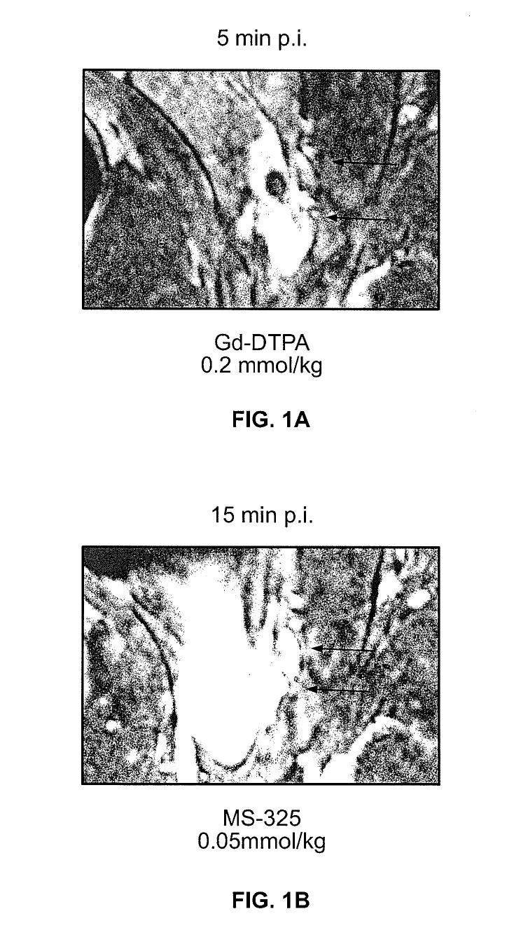

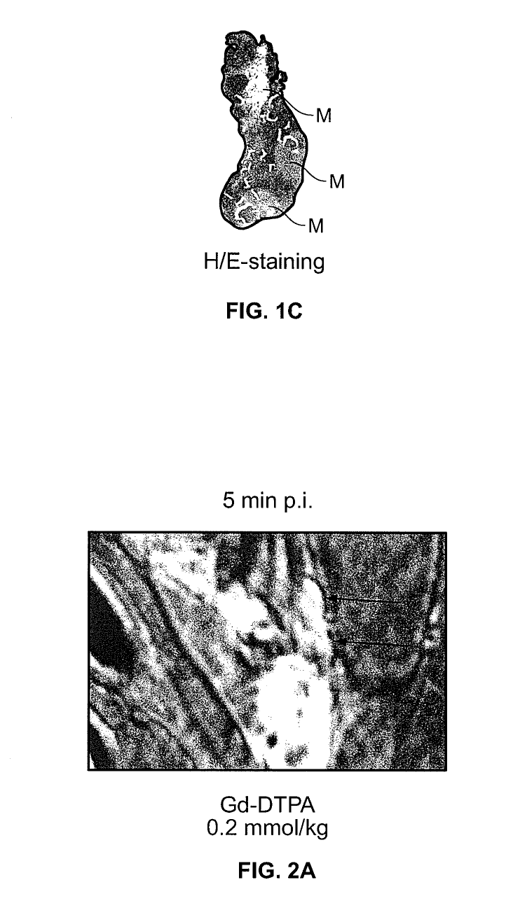

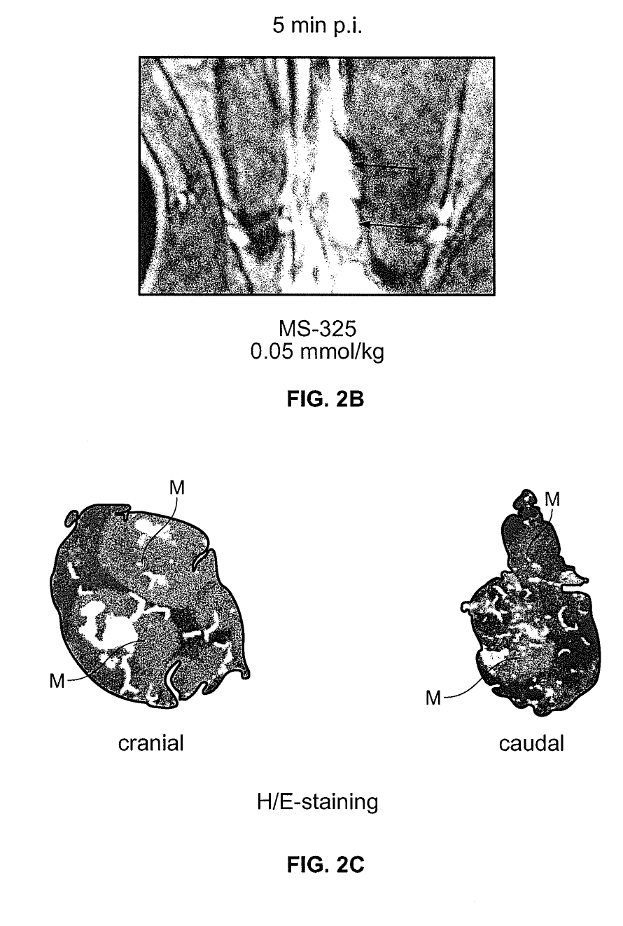

FIGS. 1 and 2 shows representative coronal MR images of metastatic iliacal lymph nodes (arrows) 5 to 15 min after intravenous injection of 0.2 mmol Gd/kg body weight of Gd-DTPA or 0.05 mmol Gd/kg body weight of MS-325. A bright and homogeneous enhancement is demonstrated in the functional lymph node tissue after injection of MS-325, while the metastases remains dark. The detection of lymph node metastases was possible and was confirmed by the microscopic examination of the dissected and histopathologically stained nodes.

### Example 2

### Enhancement of Benign Enlarged Lymph Nodes After Single Intravenous Injection of MS-325

Objectives:

The purpose of this study was to demonstrate the lymph node enhancement of enlarged popliteal lymph nodes after intravenous injection of the contrast agent MS-325.

Materials and Methods.

Animal models:

All experimental protocols were performed in accordance with applicable regulations governing animal experiments.

Female guinea pigs (370-450 g, n=3) had their lymph nodes stimulated by egg yolk emulsion (0.1 mL) intramuscularly in the thigh and lower legs on six days. MS-325 (0.05 mmol/kg) was administered as an i.v. bolus

MR Imaging:

MR system: Head scanner (Allegra, 1.5 Tesla; Siemens AG, Erlangen, Germany), T1-weighted sequence (T1-TSE, TR/TE 666/12 ms, slice thickness 1.1 mm, acquisition time 3:49).

Imaging and analysis: The animals were imaged prior to contrast agent administration and 1, 15, 30, 60, 90, 120, and 2440 minutes post injection. The percent signal intensity enhancement was calculated in the popliteal lymph nodes and in surrounding muscle. The ratio of signal intensity between the lymph node and the surrounding muscle was also determined.

TABLE 1
Signal enhancement in popliteal lymph nodes

| | % signal enhancement | ratio of signal intensity |
|---|---|---|
| Time post injection (min) | popliteal lymph node | lymph node to muscle |
| 0 | 0 ± 0 | 1.2 ± 0.1 |
| 1 | 87 ± 11 | 1.6 ± 0.1 |
| 15 | 67 ± 11 | 1.4 ± 0.1 |
| 30 | 57 ± 6 | 1.4 ± 0.1 |
| 60 | 45 ± 6 | 1.3 ± 0.1 |
| 90 | 37 ± 4 | 1.3 ± 0.1 |
| 120 | 31 ± 6 | 1.3 ± 0.1 |
| 2440 | −8 ± 14 | 1.1 ± 0.2 |

Results:

MR imaging of guinea pigs with stimulated (enlarged) lymph nodes showed a positive and persistent enhancement of the nodes after injection of MS-325 (0.05 mmol/kg). Contrast between the lymph node and the surrounding muscle was increased. After 24 hours, signal and contrast returned to baseline levels.

[image]

### REFERENCES

1. W. M. Deserno, M. G. Harisinghani, M. Taupitz, G. J. Jager, J. A. Witjes, P. F. Mulders, C. A. Hulsbergen van de Kaa, D. Kaufmann, and J. O. Barentsz, Urinary bladder cancer: Preoperative nodal staging with ferumoxtran-10-enhanced MR imaging. Radiology 233, 449-56 (2004).
2. D. M. Koh, G. Brown, L. Temple, A. Raja, P. Toomey, N. Bett, A. R. Norman, and J. E, Husband, Rectal cancer: Mesorectal lymph nodes at mr imaging with uspio versus histopathologic findings—initial observations. Radiology 231, 91-9 (2004).
3. Y. Anzai, C. W. Piccoli, E. K. Outwater, W. Stanford, D. A. Bluemke, P. Nurenberg, S. Saini, K. R. Maravilla, D. E. Feldman, U. P. Schmiedl, J. A. Brunberg, I. R. Francis, S. E. Harms, P. M. Som, and C. M. Tempany, Evaluation of neck and body metastases to nodes with ferumoxtran 10-enhanced MR imaging: Phase III safety and efficacy study. Radiology 228, 777-88 (2003).
4. M. G Harisinghani, J. Barentsz, P. F. Hahn, W. M. Deserno, S. Tabatabaei, C. H. van de Kaa, J. de la Rosette, and R. Weissleder, Noninvasive detection of clinically occult lymph-node metastases in prostate cancer. N Engl J Med 348, 2491-9 (2003).
5. H. Kobayashi, S. Kawamoto, M. W. Brechbiel, M. Bernardo, N. Sato, T. A. Waldmann, Y. Tagaya, and P. L. Choyke, Detection of lymph node involvement in hematologic malignancies using micromagnetic resonance lymphangiography with a gadolinum-labeled dendrimer nanoparticle. Neoplasia 7, 984-91 (2005).
6. Z. Zhou, J. Guo, H. Yu, B. Zhu, C. Liu, X. Jiang, M. Zhang, and J. Chen, Comparison of gd [dtpa-bis(2-aminoethoxy)ethane]polymeric contrast agent with gadodiamide injection for interstitial MR lymphography: Experimental study with rabbits. J Magn Reson Imaging 22, 361-7 (2005).
7. G Staatz, E. Spuntrup, B. Klosterhalfen, B. Misselwitz, D. Pfluger, R. W. Gunther, and A. Bucker, High-resolution T1-weighted MR-lymphography of inguinal lymph nodes after interstitial application of gadomer-17 in animal experiments. Rofo 177, 968-74 (2005).
8. N. Tsuda, T. Tsuji, and N. Kato, Interstitial magnetic resonance lymphography using gadolinium-ethoxybenzyl-diethylenetriamine pentaacetic acid in rabbits with lymph node metastasis. Invest Radiol 40, 306-12 (2005).
9. R. W. Nason, M. G. Torchia, C. M. Morales, and J. Thliveris, Dynamic MR lymphangiography and carbon dye for sentinel lymph node detection: A solution for sentinel lymph node biopsy in mucosal head and neck cancer. Head Neck 27, 333-8 (2005).
10. H. Kobayashi, S. Kawamoto, Y. Sakai, P. L. Choyke, R. A. Star, M. W. Brechbiel, N. Sato, Y. Tagaya, J. C. Morris, and T. A. Waldmann, Lymphatic drainage imaging of breast cancer in mice by micro-magnetic resonance lymphangiography using a nano-size paramagnetic contrast agent. J Natl Cancer Inst 96, 703-8 (2004).
11. C. U. Herbom, F. M. Vogt, T. C. Lauenstein, M. Goyen, O. Dirsch, C. Corot, J. F. Debatin, and S. G: Ruehm, Assessment of normal, inflammatory, and tumor-bearing lymph nodes with contrast-enhanced interstitial magnetic resonance lymphography: Preliminary results in rabbits. J Magn Reson Imaging 18, 328-35 (2003).
12. M. Shimada, K. Yoshikawa, T. Suganuma, H. Kayanuma, Y Inoue, K. Ito, A. Senoo, and S. Hayashi, Interstitial magnetic resonance lymphography: Comparative animal study of gadofluorine 8 and gadolinium diethylenetriamine-pentaacetic acid. J Comput Assist Tomogr 27, 641-6 (2003).
13. H. Kobayashi, S. Kawamoto, R. A. Star, T. A. Waldmann, Y. Tagaya, and M. W. Brechbiel, Micro-magnetic resonance lymphangiography in mice using a novel dendrimer-based magnetic resonance imaging contrast agent. Cancer Res 63, 271-6 (2003).
14. C. U. Herbom, T. C. Lauenstein, F. M. Vogt, R. B. Lauffer, J. F. Debatin, and S. G. Ruehm, Interstitial MR lymphography with MS-325: Characterization of normal and tumor-invaded lymph nodes in a rabbit model. AJR Am J Roentgenol 179, 1567-72 (2002).
15. M. G. Torchia and B. Misselwitz, Combined MR lymphangiography and MR imaging-guided needle localization of sentinel lymph nodes using gadomer-17. AJR Am J Roentgenol 179, 1561-5 (2002).
16. B. Misselwitz, H. Schmitt-Willich, M. Michaelis, and J. J. Oellinger, Interstitial magnetic resonance lymphography using a polymeric T1 contrast agent: Initial experience with gadomer-17. Invest Radiol 37, 146-51 (2002).
17. G. Staatz, E. Spuntrup, A. Buecker, B. Misselwitz, and R. W. Gunther, T1-weighted MR-lymphography after intramammary administration of gadomer-17 in pigs. Rofo 174, 29-32 (2002).
18. G Staatz, C. C. Nolte-Ernsting, A. Bucker, B. Misselwitz, S. Grosskortenhaus, D. Pfluger, and R. W. Gunther, Interstitial T1-weighted MR lymphography with use of the dendritic contrast agent gadomer-17 in pigs. Rofo 173, 1131-6 (2001).
19. G. Staatz, C. C. Nolte-Ernsting, G. B. Adam, S. Grosskortenhaus, B. Misselwitz, A. Bucker, and R. W. Gunther, Interstitial T1-weighted MR lymphography: Lipophilic perfluorinated gadolinium chelates in pigs. Radiology 220, 129-34 (2001).
20. S. G. Ruehm, C. Corot, and J. F. Debatin, Interstitial MR lymphography with a conventional extracellular gadolinium-based agent: Assessment in rabbits. Radiology 218, 664-9 (2001).
21. Y. Fujimoto, Y. Okuhata, S. Tyngi, Y. Namba, and N. Oku, Magnetic resonance lymphography of profundus lymph nodes with liposomal gadolinium-diethylenetriamine pentaacetic acid. Biol Pharm Bull 23, 97-100 (2000).
22. L. Harika, R. Weissleder, K. Poss, and M. I. Papisov, Macromolecular intravenous contrast agent for MR lymphography: Characterization and efficacy studies. Radiology 198, 365-70 (1996).
23. B. Misselwitz, J. Platzek, and H. J. Weinmann, Early MR lymphography with gadofluorine M in rabbits. Radiology 231, 682-8 (2004).
24. E. M. Delfaut, J. Beltran, G Johnson, J. Rousseau, X. Marchandise, and A. Cotten, Fat suppression in MR imaging: Techniques and pitfalls. Radiographics 19, 373-82 (1999).
25. G. M. Glazer, B. H. Gross, L. E. Quint, I. R. Francis, F. L. Bookstein, and M. B. Orringer, Normal mediastinal lymph nodes: Number and size according to American Thoracic Society Mapping. AJR Am J Roentgenol 144, 261-5 (1985).

A number of embodiments of the invention have been described. Nevertheless, it will be understood that various modifications may be made without departing from the spirit and scope of the invention. Accordingly, other embodiments are within the scope of the following claims.

What is claimed is:

1. A method for determining the presence or absence of a primary or metastatic cancer in a region of the lymph system comprising:
(a) optionally preselecting a region of the lymphatic system of a mammal to image;
(b) optionally obtaining a T1-weighted MR image of said region;
(c) intravascularly injecting the mammal with an MR contrast agent, or a pharmaceutically acceptable salt or derivative thereof, wherein said MR contrast agent is selected from Gd-BOPTA, Gd-EOB-DTPA, MP-2269, and B-22956/1, or wherein said MR contrast agent comprises a phosphodiester moiety, a PPBM, and a paramagnetic metal chelate and wherein the contrast agent is capable of binding to a plasma protein; and
(d) obtaining a T1-weighted MR image of said region of the lymphatic system, wherein said determination of the presence or absence of said primary or metastatic cancer is based on an evaluation of the signal intensity in said region of the lymphatic system.

2. The method of claim 1, wherein the signal intensity is evaluated by comparing an image obtained in step (d) with the pre-contrast agent image obtained in step (b).

3. The method of claim 1, further comprising obtaining a fat-suppressed T1-weighted MR image of the region in (d).

4. The method of claim 1, 2, or 3, wherein two or more 2D image planes of the region are examined in order to determine the presence or absence of the primary or metastatic cancer.

5. The method of claim 1, wherein said plasma protein is human serum albumin.

6. The method of claim 1, wherein said mammal is human.

7. The method of claim 1, wherein said region is one or more lymph nodes, vessels, ducts, channels or combinations thereof.

8. The method of claim 7, wherein said one or more lymph nodes, vessels, ducts, channels or combinations thereof is located in the iliac, lumbar, or inguinal region of said mammal.

9. The method of claim 7, wherein said one or more lymph nodes, vessels, ducts, channels or combinations thereof is located in the popliteal region of said mammal.

10. The method of claim 7, wherein said one or more lymph nodes, vessels, ducts, channels or combinations thereof is located in the axillary region of said mammal.

11. The method of claim 7, wherein said one or more lymph nodes, vessels, ducts, channels or combinations thereof is located in the mesenteric region of said mammal.

12. The method of claim 7, wherein said one or more lymph nodes, vessels, ducts, channels or combinations thereof is located in the cervical and/or neck region of said mammal.

13. The method of claim 7, wherein said one or more lymph nodes, vessels, ducts, channels, or combinations thereof is located in the thoracic region of said mammal.

14. The method of claim 1, wherein said PPBM is selected from alkyl, cycloalkyl, heteroalkyl, heterocyclyl, aryl, alkaryl, and aralkyl groups having from 1 to 25 carbon atoms, wherein said groups can be optionally substituted with 1 to 5 alkyl, aryl, heteroalkyl, cycloalkyl, heterocycly, alkoxy, hydroxyl, and halo groups.

15. The method of claim 14, wherein said PPBM is selected from linear or branched alkyl groups optionally substituted with one or more alkyl, aryl, alkoxy or hydroxyl groups; cycloalkyl groups optionally substituted with one or more alkyl, aryl, alkoxy or hydroxyl groups; and aryl groups optionally substituted with one or more alkyl, aryl, alkoxy or hydroxyl groups.

16. The method of claim 1, wherein said PPBM is covalently conjugated through a phospho-ester linkage to the phosphodiester moiety of the MR contrast agent.

17. The method of claim 1, wherein said paramagnetic metal chelate is selected from DTPA, DOTA, DO3A, and NOTA.

18. The method of claim 1, wherein the MR contrast agent has the following formula:
[Chel]-[Lₘ-{BHEM-PPBM}ₚ]_{q},
or pharmaceutically acceptable salts or derivatives thereof,
wherein m, p, and q are, independently, from 1 to 5;
wherein said [Chel] is a paramagnetic metal chelate selected from the group consisting of: 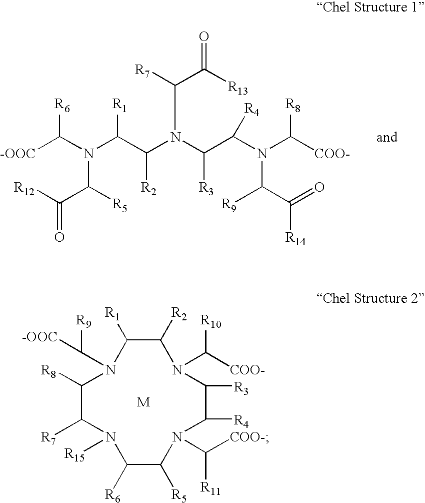
wherein at least one of said R₁-R₁₁ is -[Lₘ-{BHEM-PPBM}ₚ] and the R₁-R₁₁ groups that are not -[Lₘ-{BHEM-PPBM}ₚ] are selected from hydrogen and C1-C4 alkyl;
wherein R₁₂, R₁₃, and R₁₄ can be the same or different and are selected from the group consisting of O⁻, and NH₂;
wherein R₁₅ is H, CH₂CH(OH)CH₃, hydroxyalkyl, or CH₂COR₁₂;
wherein said M is a paramagnetic metal ion selected from the group consisting of Gd(III), Fe(III), Mn(II), Mn(III), Cr(III), Cu(II), Dy(III), Tb(III), Ho(III), Er(III), and Eu(III);
wherein said L is a linker;
wherein said BHEM is said phosphodiester moiety; and
wherein said PPBM is a plasma protein binding moiety.

19. The method of claim 1, wherein said contrast agent is selected from the following: MS-325, MS-315, MS-317, MS-322, MS-323, MS-326, MS-327, and MS-328.

20. The method of claim 1, wherein said contrast agent is MS-325.

21. The method of claim 1, wherein said MR image is obtained at a period of time between 1 minute and 24 hours after injection of said contrast agent.

21. The method of claim 1, wherein said MR image is obtained at a period of time between 5 minutes and 2 hours after injection of said contrast agent.

22. The method of claim 1, wherein said intravascular injection is in a vein.

23. The method of claim 1, wherein said intravascular injection is in an artery.

24. A method for determining whether or not to perform a biopsy of a lymph node of a mammal comprising
(a) optionally preselecting a region of the lymphatic system of a mammal to image;
(b) optionally obtaining a T1-weighted MR image of said region;
(c) intravascularly injecting the mammal with an MR contrast agent or a pharmaceutically acceptable salt or derivative thereof, wherein said MR contrast agent is selected from Gd-BOPTA, Gd-EOB-DTPA, MP-2269, and B-22956/1, or wherein said MR contrast agent comprises a phosphodiester moiety, a PPBM, and a paramagnetic metal chelate and wherein the contrast agent is capable of binding to a plasma protein;
(d) obtaining a T1-weighted MR image of said region of the lymphatic system, wherein said determination of whether or not to perform a biopsy is based on an evaluation of the signal intensity in said region of the lymphatic system.

25. The method of claim 24, further comprising:
(e) determining an appropriate location to biopsy based on the evaluation of the signal intensity in said region in the MR image of (d).

26. The method of claim 24, further comprising evaluating the signal intensity in said region in the MR image of (d) in comparison to the signal intensity of said region in the MR image of (b).

27. The method of claim 25, further comprising: (f) optionally obtaining a fat-suppressed T1-weighted MR image of the region in (d).

28. A method for distinguishing a lymph node containing a cancerous tumor from a benign enlarged node or from a normal node comprising:
(a) optionally preselecting at least one node of the lymphatic system of a mammal to image;
(b) optionally obtaining a T1-weighted MR image of said at least one node;
(c) intravascularly injecting the mammal with an MR contrast agent, or a pharmaceutically acceptable salt or derivative thereof, wherein said MR contrast agent is selected from Gd-BOPTA, Gd-EOB-DTPA, MP-2269, and B-22956/1, or wherein said MR contrast agent comprises a phosphodiester moiety, a PPBM, and a paramagnetic metal chelate and wherein the contrast agent is capable of binding to a plasma protein;
(d) obtaining a T1-weighted MR image of said at least one node, wherein said distinguishing of a cancer-containing lymph node from a benign enlarged lymph node or from a normal node is based on an evaluation of the signal intensity of said at least one node.

29. The method of claim 28, further comprising determining the size of said at least one lymph node relative to a predetermined size criterion for that anatomical region.

30. The method of claim 28, further comprising:
(e) obtaining a fat suppressed T1-weighted MR image of the at least one node in (d).

31. A method for determining the presence or absence of elephantiasis (parasitic worm infection) in a region of the lymphatic system of a mammal comprising:
(a) optionally preselecting a region of the lymphatic system of a mammal to image;
(b) optionally obtaining a T1-weighted MR image of said region;
(c) intravascularly injecting the mammal with an MR contrast agent or a pharmaceutically acceptable salt or derivative thereof, wherein said MR contrast agent is selected from Gd-BOPTA, Gd-EOB-DTPA, MP-2269, and B-22956/1, or wherein said MR contrast agent comprises a phosphodiester moiety, a PPBM, and a paramagnetic metal chelate and wherein the contrast agent is capable of binding to a plasma protein;
(d) obtaining a T1-weighted MR image of said region of the lymphatic system, wherein said determination of the presence or absence of said elephantiasis is based on an evaluation of the signal intensity in said region of the lymphatic system.
